(19) United States
(12) Patent Application Publication
(10) Pub. No.: US 2021/0283388 A1
(43) Pub. Date: Sep. 16, 2021

(54) ACTIVE SUBSTANCE PUNCH WITH AN INSERT FOR SUDDEN APPLICATION

(71) Applicant: LTS LOHMANN THERAPIE-SYSTEME AG, Andernach (DE)

(72) Inventor: Dieter PAULUKAT, Andernach (DE)

(21) Appl. No.: 17/199,341

(22) Filed: Mar. 11, 2021

(30) Foreign Application Priority Data
Mar. 11, 2020 (DE) 102020203106.1

(51) Int. Cl. A61M 37/00

(52) U.S. Cl. CPC A61M 37/0069; A61M 2202/06; A61M 2205/33; A61M 2205/12

(57) ABSTRACT

The present invention relates to an active substance punch containing a pressing device for use in the intradermal application of active substances, wherein the pressing device has an applicator arranged on at least one support which has at least two or more rods, wherein an insert having a perforated plate with at least two holes which are filled level with a solid active substance, is provided, each hole corresponds to a rod and each rod releases the active substance suddenly.

The present invention relates to an active substance punch with an insert for sudden, intradermal application.

The piercing or introduction of small drug rods is described generically for example in DE3200763A, which concerns a needleless device.

The skin is formed of a number of layers. The outermost skin layer, the stratum corneum, has known barrier properties in order to prevent the introduction of foreign substances into the body and an escape of endogenous substances from the body. The stratum corneum, which is a complex structure formed from compacted keratotic cell residues with a thickness of approximately 10-30 micrometres, to this end forms a waterproof membrane to protect the body. The natural impermeability of the stratum corneum prevents the administration of most pharmaceutical active substances and other substances via the skin barrier within the scope of a transdermal application form.

Active substances may be applied at an application site by means of a transdermal or intradermal application and may be introduced suddenly by means of a compressive force in order to penetrate the stratum corneum. Other systems are patches or microneedles, for example.

Particularly in crisis areas or in areas with little to no infrastructure, there is a need for robust and for the most part complication-free application for rapid administration of drugs, such as anaesthetics, vaccines, and many others.

The object of the present invention is therefore to achieve an intradermal application by means of an active substance punch which advantageously may be used multiple times and allows for reliable application.

Surprisingly, an active substance punch with an exchangeable insert is able to reliable and efficiently administer mass immunisations, for example, or to quickly apply active substances such as anaesthetics or vaccines for major incidents where there are many injured or impaired individuals, or for epidemics.

This object is therefore achieved in accordance with the invention by an active substance punch according to claim 1, specifically:

an active substance punch comprising a pressing device for use in the intradermal application of active substances, the pressing device having an applicator arranged on at least one support which has at least two or more rods, characterised in that an insert having a perforated plate with at least two holes (L1, L2, . . . ), which are filled level with a solid active substance, is provided, each hole corresponds to a rod (S1, S2, . . . ) and each rod releases the active substance suddenly.

It is particularly advantageous that the active substance may be introduced under the skin as a solid substance and is delivered into the bloodstream over a long period of time. This advantageously makes it possible to introduce high doses of active substances and to achieve a long-term effect.

The exchange of an insert advantageously allows an active substance punch to be used multiple times over. The perforated card may particularly advantageously be filled level with a solid substance in the holed, similarly to a blister pack. To this end, the insert may be provided with a protective film or release film, which may or may not be removed prior to the insertion into the active substance punch.

The invention therefore relates to an active substance punch comprising a pressing device for use in the intradermal application of active substances, the insert being exchangeable. In accordance with the invention the insert may be changed after use.

The active substance punch has an insertion device for an insert. The insert is preferably latched in place after insertion, such that the rods (S1, S2, . . . ) correspond to the holes (L1, L2, . . . ) and the rods (S1, S2, . . . ) may enter the holes (L1, L2, . . . ) with an accurate fit.

The rods may be designed in accordance with the invention as needles, for example, and after entering the holes it is preferred that the needles do not touch the skin and do not protrude beyond the skin-side surface of the insert. This measure according to the invention in particular allows pharmacologically and hygienically safe treatment of different patients or test subjects. The rods may be formed for example from steel or titanium and may be smooth, flat, round or pointed at the proximal end.

In accordance with the invention it is preferred that the insert has a perforated plate with holes filled with active substance, which holes are level or planar with top and bottom side of the perforated plate or the holes filled with the active substance terminate flush and level with the top and bottom side of the perforated plate.

It is further preferred that the perforated plate of the insert has a thickness of at most 10 mm, preferably 5 mm, particularly preferably 1 mm or less, and the holes have a diameter of at most 2 mm, preferably 1.5 mm, particularly preferably 1 mm or less.

The invention therefore also relates to an insert having a perforated plate with holes filled with active substance, which holes are level or planar with the top and bottom side of the perforated plate.

The perforated plate is preferably made of aluminium, plastic, steel or titanium or other break-resistant/tough materials. Titanium is particularly preferred.

The term “intradermal application (synonym: “intracutaneous application”) in accordance with the invention describes the administration of active substances into the skin and requires a sudden infiltration of the active substances into the skin by means of the active substance punch according to the invention.

The term “solid active substance” in the sense of this invention includes any solid active substances, but preferably drugs, particularly preferably active substances or drugs in crystalline or pourable or porous form. For example, such preferred crystalline forms may be produced by means of dicarboxylic acids or dicarbonates, in particular oxalic acid or oxalates. Furthermore, the active substances may be present in any solid formulation. Relevant auxiliaries and additives are known to a person skilled in the art.

Within the scope of this invention an active substance punch is a suitable system which contains a pressing device which causes an active substance to be provided for administration on the skin and to be applied intradermally.

The active substance punch, in the pressing device, has an applicator for example in the form of a knob or plunger, in particular in the form of a trigger device which is controlled electrically or mechanically, for example in the form of a push-button. The trigger device may have a piston or spring, for example, such that the generated compressive force is transferred via the support to the rods and releases the active substance suddenly from the holes into the perforated plate.

Besides the manual triggering, the trigger device may preferably comprise a spring, such that a piston thrust with sufficient energy may be provided. The piston may be of any form and any kind.

The invention also relates to a kit or cassette containing an active substance punch according to the invention with one or more inserts.

The invention also relates to a method or use for performing an intradermal application of active substances comprising
a) fixing an active substance punch according to the invention against the skin,
b) penetrating an active substance into the skin, wherein an insert having a perforated plate with at least two holes (L1, L2, . . . ), which are filled level with a solid active substance, is provided, each hole corresponds to a rod (S1, S2, . . . ) and each rod releases the active substance suddenly.

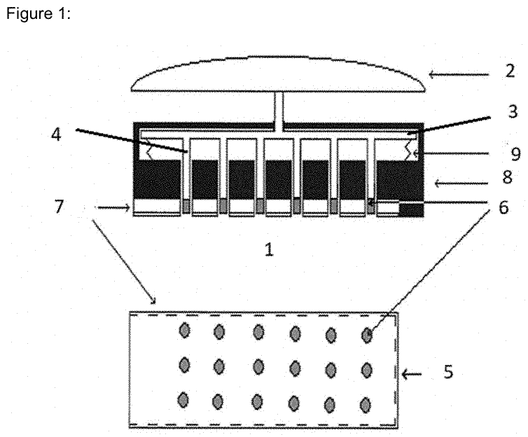

### FIGURE 1

Principal structure of an active substance punch with an insert which has a perforated plate with holes filled with active substance.

### KEY TO THE FIGURE

1. active substance punch (overall view)
2. applicator (for example knob, plunger, as shown)
3. support
4. rod (S1, S2, . . . )
5. perforated plate
6. holes filled with active substance (L1, L2, . . . )
7. insertion
8. housing
9. spring

1.-12. (canceled)

13. An active substance punch comprising a pressing device for use in the intradermal application of active substances, the pressing device having an applicator arranged on at least one support which has at least two or more rods, wherein an insert having a perforated plate with at least two holes which are filled level with a solid active substance, is provided, each hole corresponds to a rod and each rod releases the active substance suddenly.

14. The active substance punch according to claim 13, wherein the insert is exchangeable.

15. The active substance punch according to claim 13, wherein the active substance punch has an insertion device for an insert.

16. The active substance punch according to claim 13, wherein the insert has a perforated plate with holes filled with active substance, which holes are level or planar with the top and bottom side of the perforated plate.

17. The active substance punch according to claim 13, wherein the insert has a thickness of at most 10 mm and the holes have a diameter of at most 2 mm.

18. The active substance punch according to claim 13, wherein the insert has a thickness of at most 5 mm and the holes have a diameter of at most 1.5 mm.

19. The active substance punch according to claim 13, wherein the insert has a thickness of at most 1 mm, and the holes have a diameter of at 1 mm.

20. The active substance punch according to claim 13, wherein the applicator comprises a knob or comprises a trigger device which is controlled electrically or mechanically.

21. The active substance punch according to claim 13, wherein the active substance is present in crystalline form.

22. An insert for use in the intradermal application of active substances, having a perforated plate with holes filled with active substance, which holes are level or planar with the top and bottom side of the perforated plate.

23. The insert according to claim 22, wherein the insert has a thickness of at most 10 mm and the holes have a diameter of at most 2 mm.

24. The insert according to claim 22, wherein the insert has a thickness of at most 5 mm and the holes have a diameter of at most 1.5 mm.

25. The insert according to claim 22, wherein the insert has a thickness of at most 1 mm, and the holes have a diameter of at 1 mm.

26. The insert according to claim 22, wherein the active substance is present in crystalline form.

27. The insert according to claim 22, wherein the perforated plate is formed from steel or titanium.

28. A kit or cassette comprising an active substance punch according to claim 13.

29. A kit or cassette comprising one or more inserts according to claim 22.

30. A kit or cassette comprising an active substance punch according to claim 13 and having a perforated plate with holes filled with active substance, which holes are level or planar with the top and bottom side of the perforated plate.
